(19) United States
(12) Patent Application Publication
(10) Pub. No.: US 2023/0045428 A1
(43) Pub. Date: Feb. 9, 2023

(54) System And Method For Treating Microorganisms

(71) Applicants: Brochier Technologies, Villeurbanne (FR); Centre National de la Recherche Scientifique, Paris (FR); Universite Claude Bernard Lyon 1, Villeurbanne (FR); Institut National De La Sante Et De La Recherche Medicale, Paris (FR); Ecole Normale Superieure De Lyon, Lyon (FR)

(72) Inventors: Chantal GUILLARD, Thil (FR); Christophe GILBERT, Thil (FR); Cédric BROCHIER, Lyon (FR); Laure PERUCHON, Villeurbanne (FR); Lina LAMAA, Vaulx-en-Velin (FR); Davide LORITO, Villeurbanne (FR)

(73) Assignees: Brochier Technologies, Villeurbanne (FR); Centre National de la Recherche Scientifique, Paris (FR); Universite Claude Bernard Lyon 1, Villeurbanne (FR); Institut National De La Sante Et De La Recherche Medicale, Paris (FR); Ecole Normale Superieure De Lyon, Lyon (FR)

(21) Appl. No.: 17/790,588

(22) PCT Filed: Dec. 20, 2020
(86) PCT No.: PCT/FR2020/052611

§ 371 Date: Jul. 1, 2022

(30) Foreign Application Priority Data
Jan. 15, 2020 (FR) 2000384

(51) Int. Cl. A61L 9/20; A61L 2/26; A61L 2/10; A61L 2/08; A61L 2/238; C02F 1/72; C02F 1/32; B01D 53/88; B01D 53/00

(52) U.S. Cl. CPC A61L 9/205; A61L 2/26; A61L 2/10; A61L 2/088; A61L 2/238; C02F 1/725; C02F 1/32; B01D 53/885; B01D 53/007; A61L 2202/11; A61L 2209/12; A61L 2209/14; C02F 2305/10; C02F 2303/04; B01D 2255/802; B01D 2257/91; B01D 2259/804

(57) ABSTRACT

A system for the treatment of microorganisms includes: a textile web having optical fibers in warp and/or weft woven with binding threads in warp and/or weft, each of the optical fibers having invasive alterations along the fiber and allowing the emission of light propagating in the fiber at these alterations; a light source arranged opposite one or both free ends of the optical fibers. The textile web includes metallic warp and/or weft threads woven with the binding threads, the metallic threads being based on a metal having a negative effect on the growth of microorganisms. The light source generates a light beam having at least one wavelength in the visible or ultraviolet spectrum.

### TECHNICAL FIELD

The invention relates to the field of contaminated media treatment and relates more particularly to a system and a method for the treatment of microorganisms, for example to reduce the quantity of microorganisms in a liquid or gaseous medium.

### PRIOR ART

The generic term microorganism includes all microscopic living beings such as bacteria, fungi, parasites, and viruses. Different qualifications may be attributed to these microorganisms according to their effect on human beings, their mode of development, etc. We distinguish, for example, the so-called pathogenic microorganisms (designated under the name of microbes in everyday language) capable of causing disorders organic, so-called cultivable microorganisms, etc. Of course, several qualifications may be attributed to the same microorganism. For example, the bacterium Escherichia Coli is in particular considered as a culturable and pathogenic microorganism, whereas a virus is generally considered as a non-culturable pathogen.

In the particular case of pathogenic and cultivable microorganisms such as the bacterium Escherichia Coli (E. Coli), various antimicrobial solutions are developed for the purpose of slowing down or preventing the growth of these microorganisms.

In particular, it is known that UV radiation, certain metals, and certain semiconductor oxides, when implemented separately, exhibit antimicrobial effects, with different modes of action and different conditions of action.

Ultraviolet (UV) radiation causes molecular alterations in living cells, to a greater or lesser extent depending on their wavelength. In particular, we distinguish:
UV type A (UV-A), with wavelengths between 315 nm and 400 nm, which cause a molecular alteration of living cells;
type B UV (UV-B), with wavelengths ranging from 280 nm to 315 nm, more damaging than UV-A for living cells; and
type C UV (UV-C), with wavelengths between 100 nm and 280 nm and which are very harmful, even fatal for humans, but which have the advantage of having a very good germicidal action.

Briefly, at the level of known mechanisms, the aromatic cycles of the (A, G, T, C) bases of the DNA molecule absorb the energy of photons associated with a wavelength between 230 and 290 nm (UV-C and low wavelength UV-B). The energy absorbed at two adjacent pyrimidines (C or T) provides the energy necessary for the formation of a covalent bond between these two bases, essentially forming cyclobutane dimers of pyrimidines (cyclobutane pyrimidine dimer, CPD) and the pyrimidines (6-4) pyrimidone (6-4 PP) which then cause a distortion of the DNA double helix and in particular block the progression of replicative polymerases. In the absence of repair, there is a risk of inserting an incorrect base (mutation) in the next replicative cycle and depending on the number of mutations and their importance, a deleterious effect on the cell may be observed.

As far as UVA rays are concerned, they are only weakly absorbed by DNA bases but they can excite cellular chromophores, called photosensitizers, which return to their ground energy state by dissipation of heat or emission of photons (this is the phenomenon of fluorescence) but can also undergo a transition to a more stable energy state called the triplet state. This triplet plays a key role in inducing UV-A damage by reacting directly with other molecules, such as DNA bases, (type I photosensitization) or by transferring its energy to oxygen molecules (type II photosensitization), thus leading to the formation of reactive oxygen species (ROS): singlet oxygen (¹O₂) or superoxide anion (O₂ ^{⋅-}). In addition, the hydroxyl radical (^{⋅}OH) may be formed in the presence of transition metals from hydrogen peroxide (H₂O₂) itself resulting from disproportionation of the superoxide anion. The accumulation of ROS in the cell can cause direct damage to all cellular components including protein oxidation and nucleic acid alteration, in particular DNA helix breaks (single or double-stranded).

Among the semiconductor oxides, mention may be made of titanium dioxide (TiO₂) known for its photocatalytic properties contributing in particular to the inactivation of bacteria, viruses, and molds. In practice, a thin film based on TiO₂ is deposited or formed on a substrate. Activation of the photocatalyst by irradiation, for example under ultraviolet (UV) radiation, produces an oxidation-reduction reaction generating “electron-hole” pairs. These “electron-hole” pairs react with the oxygen and humidity contained in the medium, such as air or water, to produce free radicals that are harmful to microorganisms. For example, document FR2910341 of the Applicant describes the deposition of a layer of TiO₂ on the optical fibers configured to emit UV radiation.

Among the metals having an antibacterial property, mention may be made of silver (Ag). Silver ions (Ag+) have the ability to penetrate the very heart of bacteria, and inactivate their vital enzymes or generate hydrogen peroxide, which inevitably leads to bacterial death. On the other hand, and unlike titanium dioxide, silver does not eliminate the bacterial residues that are generated. Copper (Cu) may also be mentioned for its antimicrobial properties. In water, the ability of bacteria to reproduce may be greatly affected depending on the amount of copper ions present. In practice, copper ions have been observed to attack the cell membrane of bacteria, asphyxiate the bacteria, then attack the genomic material (DNA) of the bacteria, inducing its death.

Combining metals, such as silver or copper, and titanium dioxide in different formulations in the form of composite powders or thin composite films, for the purpose of improving the photocatalytic activity of titanium dioxide TiO₂, was considered. In particular, it has been shown that by promoting the separation of charges, silver reduces the recombination of the photo-generated “electron-hole” pairs. Thus, copper or silver particles may be incorporated in the form of a thin film combined with particles of titanium dioxide TiO₂. The whole is then deposited on a substrate.

Furthermore, to increase the effectiveness of the action of the metal on the bacteria, one solution consists in increasing the contact surface of the metal surface with the bacterial cells. For example, one solution to limit the size of the substrate consists of creating rough areas on the thin film to trap the bacteria within these rough areas, thus increasing the contact surface.

However, this thin film solution remains complex to implement since it requires controlling various factors related to the process to deposit the film on the substrate, such as the size of the metal particles to be incorporated to fill the interstices between the TiO₂particles, the amount of gas supplied, etc. . . . In addition, peeling and premature depletion of copper particles constitute a major problem encountered in solutions based on thin films. Furthermore, in most solutions, UV radiation is generally provided by an external light source, such as a lamp or several lamps placed at a certain distance from the substrate in order to be able to activate a larger area of the film. This solution induces a higher cost and non-optimal efficiency. Another equally complex and costly solution consists of depositing the antimicrobial film on a glass substrate that makes it possible to capture the light emitted by the sun and to convey it to activate the photocatalytic particles.

### DISCLOSURE OF THE INVENTION

This invention thus proposes an alternative solution that is easy to implement, compact, does not require complex manufacturing steps, and that nevertheless has an effect on the activity of microorganisms, making it much better in comparison to existing solutions.

This invention aims in particular to propose an alternative solution that makes it possible to prevent the growth of microorganisms, for example pathogenic or non-pathogenic cultivable microorganisms, present in a medium, by reducing or slowing down the activity of these microorganisms, by inactivation or inhibition of these microorganisms, by elimination, or even by reducing the quantity of these microorganisms in the medium.

The solution of the invention has in particular the following advantages:
rapid and effective action on organic contaminants, but also on microorganisms such as germs;
more compact;
malleable and modular;
less complex manufacturing compared to chemical deposition of metal particles;
more durable.

The invention therefore proposes a textile web comprising optical fibers in warps and/or weft woven with binding threads in warp and/or weft. Each optical fiber has invasive alterations along the fiber, and allows the emission, through these alterations, of light propagating in the fiber. The textile web further comprises metallic warp and/or weft threads also woven with binding threads, which may be identical to or distinct from those associated with the optical fibers. The metal threads are based on a metal having a negative effect on the growth of microorganisms, preferably based on a metal having antimicrobial properties.

The negative effect on the growth of microorganisms can in particular result in the reduction of the activity of at least the targeted microorganisms in the treated medium, or their inactivation (or inhibition), or the reduction in the quantity of these targeted microorganisms present in the medium treated.

This textile web is intended to be implemented in a system for treating microorganisms, such as an antimicrobial system, therefore comprising at least one textile web as defined above, as well as a light source arranged opposite one of the two free ends of the optical fibers and able to generate a light beam also having a negative effect on the growth of microorganisms. In practice, the light beam may comprise at least one wavelength in the visible or ultraviolet spectrum. In practice, the negative effect of the textile web on the microorganisms is obtained with a light beam preferably comprising at least one electromagnetic/light radiation of wavelengths between 100 nm and 400 nm. Advantageously, the light radiation may thus be ultraviolet radiation (i.e., in the 100 nm-400 nm spectral band) or visible-near ultraviolet radiation (i.e., in the 400 nm-500 nm spectral band).

In practice, this textile web may equally well be made in the form of a fabric, a knitted fabric, or a braided one. Generally, the luminous textile web is preferably a fabric which is composed of warp threads and weft threads arranged according to predetermined patterns that those skilled in the art will be able to determine according to the applications. Advantageously, this fabric may be obtained by a Jacquard process during which the mode of distribution of the warp and/or weft threads and the metal threads, as well as that of the optical fibers, is precisely controlled. Thus, the optical fibers and the metallic threads are advantageously woven within a textile core in a contiguous and identifiable manner. The textile core serves in particular as a support for holding the optical fibers and the metallic threads.

It is preferable for the metal threads to extend parallel to the optical fibers. The textile web can thus comprise binding threads allowing the optical fibers and metallic threads to be held within the woven textile core. These binding threads are warp threads when the optical fibers and the metallic threads are inserted into the weft, and these binding threads are weft threads when the optical fibers and the metallic threads are inserted into the warp. However, the optical fibers and the metallic threads are preferably inserted in the weft and in this case, the binding threads are warp threads. Furthermore, the textile web may advantageously have binding threads distributed over the optical fibers in a satin-type weave so as to optimize the diffusion surface of the optical fibers. The light device may have different arrangements depending on the intended applications.

The solution of this invention therefore consists of a textile web based on side-emitting optical fibers and metallic threads, all held together by weaving via binding threads. Light radiation, such as ultraviolet light, is therefore guided in a distributed manner inside the textile web thanks to the lateral emission optical fibers and is therefore conveyed to the very heart of the medium to be treated. In addition, the interstices of the textile web at the intersections of the threads constituting it, increase the contact surface of the textile web with the organisms present in the environment, and therefore optimize the action of light radiation combined with the action of the metallic threads on the targeted microorganisms. Moreover, due to the integration of antimicrobial compounds in the form of metal threads, the antimicrobial source per surface unit is able to be in greater quantity compared to solutions integrating thin metal films and therefore remains available longer. Therefore, the service life of the textile web of the invention as a treatment system is greater. Furthermore, the integration of a metal source in the form of threads avoids problems of peeling and therefore the premature depletion of the antimicrobial source.

The textile web thus formed is moreover easily manipulated and adjustable. In particular, the thickness and flexibility of such a textile web is comparable to that of a fabric. As a result, it may specifically be used as it is or be attached to supports of different shapes. For example, a simple cutting of the textile web to the desired dimensions makes it possible to produce decontamination devices of any size.

Advantageously, the metal is preferably chosen from the group comprising silver (Ag) and copper (Cu). In practice, a metal thread may consist of a single filament (monofilament) in the form of a so-called pure metal (copper or silver) thread, comprising for example 99.9% of the metal (copper or silver), and having for example a diameter substantially on the order of 10 to 300 μm. It is also possible to use a monofilament metal thread consisting of a blend of two metals based on copper and silver, for example a thread consisting of copper covered with silver or a silver thread covered with copper. The monofilament metal thread may also be in the form of a textile thread coated with a layer of metal. According to another variant, a metal thread may be composed of several filaments (multifilament) combined together via different assembly techniques. Thus, by way of example, a multifilament metal thread may be in the form of a wrapped thread or a twisted thread. In practice, a multifilament metal thread preferably comprises at least one textile thread assembled with at least one pure metal thread or a textile thread coated with a metal layer. according to any embodiment, the metal thread may comprise one or more twisted metal-based thread(s) (silver and/or copper) with one or more textile thread(s), such as polyester, polyamide or any other fiber. The metal thread thus formed may have a titration of between 50 and 1000 decitex (Dtex).

Furthermore, the light source preferably generates ultraviolet radiation of type A (UV-A) or of a wavelength between 315 nm and 400 nm. Indeed, it has been observed that the synergy of copper or silver threads combined with UV-A radiation on certain bacteria, such as Escherichia coli (E. coli), is considerably increased. Such a solution is therefore less harmful to humans, unlike solutions advocating the use of UV-C which requires precautions for use and special warnings. Preferably, the sufficient applied light intensity is 100 μW/cm².

Different assembly or weaving techniques may be implemented depending on whether it is desired to obtain a textile web having optical fibers and/or metallic threads visible on only one surface or on both surfaces of the web.

According to a variant, the textile web has two opposite visible surfaces, and optical fibers and metallic threads are visible on the two opposite surfaces of the web. In this variant, the optical fibers and the metallic threads are woven with the binding threads so as to form a fabric. The metallic threads extend parallel to the optical fibers and the fabric is formed by alternating optical fibers and metallic threads on each of its surfaces.

According to another variant, the textile web has two opposite visible surfaces, the optical fibers and the metal threads being visible on only one of the two surfaces. In other words, a specific technique of weaving metallic threads with binding threads and optical fibers with these same binding threads makes it possible to position the optical fibers and the visible metallic threads on only one and the same surface of the textile web.

In another variant, the textile web has two opposing visible surfaces, with the optical fibers visible on one surface and the metal threads visible on the other surface. In other words, a specific weaving technique of metallic threads with binding threads and optical fibers with these same binding threads makes it possible to position the visible optical fibers on only one surface of the textile thread and to position the visible metallic threads only on the other surface of the textile web.

In another variant, the textile web has two opposing visible surfaces, with optical fibers visible on only one surface and metal threads visible on both surfaces. In other words, a particular weaving technique of metallic threads with binding threads and of optical fibers with these same binding threads makes it possible to position the optical fibers so as to make them visible on only one surface of the textile web and to position the metallic threads so as to make them visible on both surfaces of the web. In other words, in this other variant, a first visible surface of the textile web comprises an alternation of optical fibers and metallic threads, and a second visible surface of the textile web comprises exclusively metallic threads.

Similarly, according to another variant, it is also possible to position the optical fibers so as to make them visible on both sides of the web and to position the metal threads so as to make them visible only on one side of the textile web.

According to another variant, the textile web may be formed from a superposition of textile layers, each textile layer comprising optical fibers and metal threads which are held together by binding threads, and which are visible on one or both surfaces of the layer, for example according to at least one of the variants disclosed above. The textile web thus has more interstices (and therefore contact surfaces) to capture/trap the target microorganisms.

According to another variant, the textile web can comprise a superposition of textile layers in which a first textile layer is formed of optical fibers held by binding threads within a textile core and a second textile layer is formed of metal threads held by binding threads within another textile core. Thus, the textile web may have alternating first and second textile layers.

According to any embodiment, it is possible to integrate photocatalytic particles into the textile web so as to increase the desired effect. The photocatalytic particles may be added in different ways to the textile web and may form a layer covering the entire textile web or only predefined areas.

For example, the photocatalytic particles may first be applied to the different components of the textile web, before weaving. Thus, the textile web may further comprise a coating layer integrating photocatalytic particles deposited on all or part of the optical fibers and/or all or part of the binding threads (warp and/or weft thread) before weaving. Preferably, the coating layer incorporating the photo-catalytic particles is deposited on the binding threads.

The photocatalytic particles may also be added after weaving the optical fibers with the binding threads. The photocatalytic particles may be deposited on the entire fabric formed by the optical fibers associated with the binding threads or on predefined zones. Thus, the textile web may further comprise a coating layer incorporating photocatalytic particles deposited on all or part of at least one of the surfaces of the fabric formed by the optical fibers woven with the binding threads. Most metal threads do not have this coating. This coating layer may be deposited in various ways, for example by bathing, padding, emulsion, spraying, printing, encapsulation, electroplating.

In practice, the photocatalytic particles are formed in a material chosen from the group comprising titanium dioxide, zinc oxide, zirconium dioxide, and cadmium sulfide. Preferably, the photocatalyst is based on titanium dioxide (TiO₂), for example TiO₂ anatase and/or rutile. In this case, the intensity of the applied light intensity is advantageously 100 μW/cm² in the wavelength range below 400 nm, so as to activate the photocatalysts.

It is also possible to provide a protective layer of silica (SiO₂) prior to coating the photocatalytic layer. In practice, the silica layer is deposited between the layer integrating the photocatalytic particles and the optical fibers and/or the binding threads. Preferably, the protective layer and the coating layer integrating the photocatalytic particles are deposited on the binding threads.

The invention also provides a method of treatment, e.g., a reduction in the activity of microorganisms in a liquid or gaseous medium, comprising:
placing the textile web as defined above in said medium; and
lighting one or both free ends of the optical fibers with said light source.

Preferably, the textile web is not enclosed in a housing or casing, even a transparent one, but brought into direct contact with the medium to be treated so that the microorganisms present in the medium to be treated may be in direct contact with the surfaces of the textile web.

In practice, the adjustment parameters such as the wavelength of the light radiation, the light intensity, the time, or the frequency of exposure, depend on the type of target microorganisms and on the medium. For example, for E. coli bacteria in a liquid medium, the textile web is immersed in the liquid medium and UV-A radiation, preferably with a wavelength between 315 and 400 nm, and an intensity of 100 μW/cm² is applied.

The application of radiation in the visible spectrum can also be envisaged when the textile web is devoid of TiO₂. An effect on the inactivation of E. Coli microorganisms has been observed specifically with light radiation generated by a white LED making it possible to obtain a light intensity at the surface of the textile on the order of 500 Cd/m². However, the time observed for full inactivation is longer compared to the application of UV-A radiation.

### BRIEF DESCRIPTION OF FIGURES

Other characteristics and advantages of the invention will become clear from the following description, given with reference to the attached drawings and which is indicative and not limiting, in which:

FIG. 1 is a perspective view of a textile web according to any embodiment of the invention;

FIGS. 2A-2G are cross-sectional views of the textile web according to different variants in the arrangement of the optical fibers and of the metallic threads;

FIG. 2A is a cross-sectional view of the textile thread according to a variant in which the optical fibers and metal threads are woven so as to be visible on both surfaces of the textile web;

FIG. 2B is a cross-sectional view of the textile thread according to another variant in which the optical fibers and the metal threads are woven in such a way as to be visible on both surfaces of the textile web;

FIG. 2C is a cross-sectional view of the textile web according to a variant in which the optical fibers and the metal threads are woven in such a way as to be visible on a single surface of the textile web;

FIG. 2D is a cross-sectional view of the textile web in another variant in which the optical fibers and metal threads are woven so as to be visible on different surfaces of the textile web;

FIG. 2E is a cross-sectional view of the textile web according to another variant in which the metal fibers are visible on both surfaces and the optical fibers are visible only on one surface of the textile web;

FIG. 2F is a cross-sectional view of the textile web according to another variant in which the optical fibres are visible on both surfaces and the metal threads are only visible on one surface of the textile web;

FIG. 2G is a cross-sectional view according to another variant in which a textile web based on optical fibres is combined with another textile based on metal threads;

FIG. 3 is a schematic representation of the textile web in use;

FIG. 4 is a graphical representation showing the antimicrobial effect of the textile web provided with copper and/or silver metal threads;

FIG. 5 is a graphical representation showing the antimicrobial effect of copper combined or not with TiO₂;

FIG. 6 is a graphical representation showing the antimicrobial effect of the textile web in a gaseous medium.

It will be noted that in these figures, the same references designate identical or similar elements, and the various structures are not to scale. Furthermore, only the essential elements for understanding the invention are shown in these figures for reasons of clarity.

### DETAILED DESCRIPTION OF THE INVENTION

The treatment solution of the invention is described below, by way of non-limiting example, in the specific case of an antimicrobial treatment. according to any embodiment of the invention, the treatment solution therefore comprises a textile web obtained by weaving optical fibers, metallic threads, and binding threads. The end of the optical fibers is coupled to a light source configured to generate UV radiation.

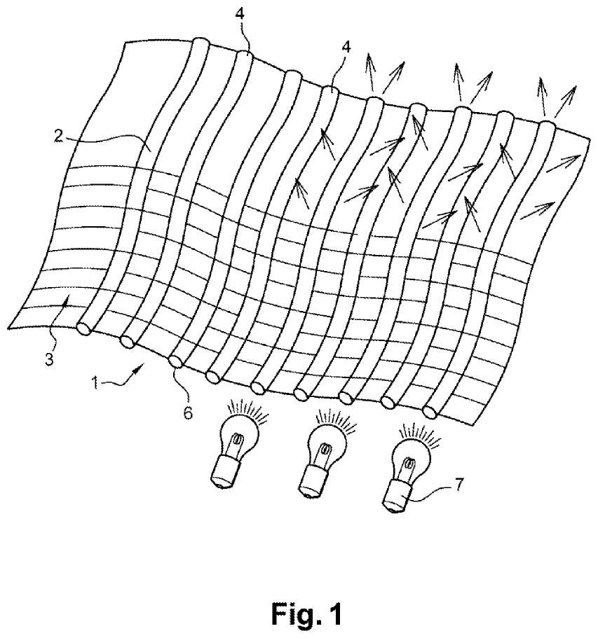

The textile web 1 according to any embodiment is illustrated in FIG. 1 and therefore incorporates side-emitting optical fibers 2 and metallic threads 4 having in particular antibacterial and/or antimicrobial properties. The optical fibers 2 and the metal threads 4 extend parallel to each other.

These optical fibers 2 and these metallic threads are arranged in warp and/or weft and are woven with binding threads 3 arranged in warp and/or weft. The ends 6 of the optical fibers 2 are intended to be arranged facing a light source 7 configured to generate ultraviolet radiation, in particular of the UV-A type.

In practice, the binding threads may be woven according to a plain-type weave which provides optimum mechanical strength and surface uniformity. Other types of weaving may be envisaged, such as satin, twill or other. The binding threads may be formed from a material chosen from the group comprising polyamide, polyester, polyethylene, and polypropylene, or any other textile fiber.

Furthermore, the optical fibers can comprise a core formed from a material chosen from the group comprising polymethyl methacrylate (PMMA), polycarbonate (PC), and cyclo-olefins (COP). In this case, the optical fibers are made of two materials and have a core covered with a sheath which may be of different nature. The optical fibers may also be formed from a material chosen from the group comprising glass, quartz, and silica. In this case, a polymer sheath may cover the optical fibers to protect them. Also, these optical fibers either have a modification of the material of the optical cladding, or invasive alterations on their outer surface, so that the light propagating in the fiber escapes from the fiber through the modified cladding or these alterations. These alterations may be carried out in various ways, including by abrasion processes, chemical attack, or by laser treatment. In addition, these alterations may be distributed progressively over the surface of the optical fibers so as to ensure homogeneous lighting. The surface density or the dimension of the alterations can thus vary from one zone to another of the web. In general, close to the light source, the surface density of alterations is low, while it increases the further one moves away from the source.

The light source 7 intended to illuminate the free ends 6 of the optical fibers 2 may be of different types and is chosen from among those capable of generating radiation including UV-A ultraviolet radiation which is not very harmful. For example, the light source 7 may be in the form of light-emitting diodes, or even comprise a collector capable of focusing natural sunlight, which comprises about 4-5% UVA, in the direction of the free ends of the optical fibers.

In order to ensure antimicrobial action, the metal threads may be based on silver or copper metal threads. The metal threads can thus be pure silver threads or pure copper threads comprising for example 99.9% silver or copper, respectively. The metal threads may also be metal-coated textile threads. The diameter of the metal threads is unimportant and depends on the weaving technique or even on the desired flexibility of the textile web. By way of example, it is possible to use textile threads coated with silver having a titer of the order of 100 Dtex, or pure copper threads having a diameter on the order of 0.1 mm.

To increase the antimicrobial effect of the textile web, it is possible, according to another embodiment, to integrate photocatalytic particles having an effectiveness on the bacterial inactivation, such as titanium dioxide (TiO₂).

For example, the photocatalytic particles may first be deposited on the optical fibers and/or the binding threads before weaving, in the form of a coating layer so as to form a sheath around each optical fiber and/or around each binding thread. The optical fibers and the metallic threads are then held together by weaving with the binding threads. To prevent premature aging of the optical fibers caused by the titanium dioxide, it is possible to provide for the deposition of a protective layer based on silica prior to the deposition of the photocatalytic layer. It is also possible to provide for the deposition of the photocatalytic layer after weaving the optical fibers and the metal threads with the binding threads. Thus, after weaving, a coating layer incorporating photocatalytic particles, as well as the intermediate layer of silica, is deposited.

Furthermore, depending on the application in which the textile web is intended to be implemented, it is possible to provide different configurations in the arrangement of the optical fibers and the metal threads.

It is in particular possible to envisage choosing to make the metallic threads and/or the optical fibers visible on the two opposite surfaces of the web or on only one of the two surfaces.

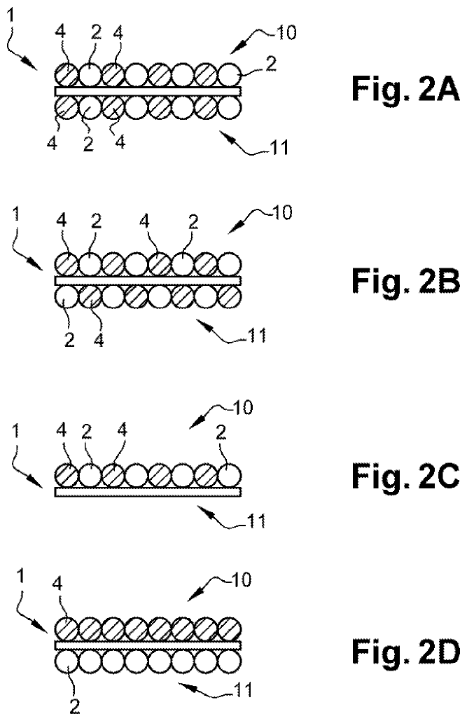

For example, the optical fibers 2 and the metallic threads 4 may be positioned so as to be visible on the two opposite surfaces 10, 11 of the textile web 1 (FIGS. 2A and 2B).

The weaving technique of the binding threads with the optical fibers and the metal threads is such that the textile web has on each of these two opposite surfaces 10, 11 an alternation of optical fibers and metallic threads. Different configurations of alternation between the optical fibers and the metallic threads may be envisaged on each of the surfaces of the textile web, such as those illustrated in FIGS. 2A and 2B. It is also possible to envisage an alternation between groups of optical fibers and groups of metal threads. In other words, each of the surfaces may comprise an alternation of optical groups and metal groups, each optical group being made up of one or more optical fibers and each metal group being made up of one or more metal threads.

The optical fibers 2 and the metal threads 4 can also be visible only on one and the same single surface 10 (FIG. 2C) of the textile web 1. In this case, the textile layer comprises only one luminous surface provided with metallic threads.

The optical fibers 2 and the metallic threads 4 can also be visible on opposite surfaces 10, 11 (FIG. 2D) of the textile web 1. Thus, the optical fibers are only visible on one side of the textile web and the metallic threads are only visible on the other side of the textile web.

Another variant consists in making the metallic threads 4 visible on the two surfaces 10, 11 of the web while the optical fibers 2 are only visible on one side of the textile web 1 (FIG. 2E), or else in making the fibers optical 2 visible on both sides 10, 11 and the metal thread 4 visible on one side of the textile web (FIG. 2F).

According to another variant, the textile web may be formed from a superposition of textile layers, each textile layer comprising optical fibers and metal threads which are held together by binding threads, and which are visible on one or both surfaces of the layer, for example according to at least one of the variants set out above. The textile web thus has more interstices (and therefore contact surfaces) to capture/trap the target microorganisms.

In another variant illustrated in FIG. 2G, a textile web based on optical fibers is superimposed onto another textile web based on metallic threads. The textile web can thus comprise a superposition of textile layers, a first textile layer 1b formed of optical fibers 2 held by binding threads and a second textile layer 1a being formed of metal threads 4 held by binding threads.

According to the same principle of the superposition of textile layers, it is possible to superimpose several textile webs, each of which may be according to any of the variants set out above.

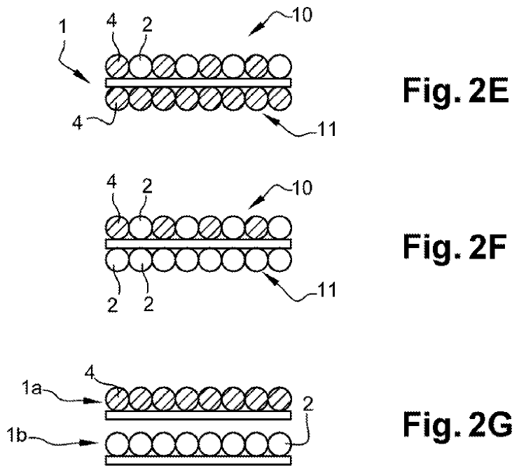

The use of such a textile web formed of optical fibers and metal thread, in particular copper, provided or not with a photocatalytic layer is illustrated in FIG. 3. The textile web 1 is represented in a simplified manner. A light source 7 is positioned facing the free ends 6 of the optical fibers 2. These may be grouped together in bundles or not. Thus, the light emitted laterally by the optical fibers 2 may be transmitted on either side of the textile web 1 perpendicular to each of these surfaces, but also inside the textile web.

Surprisingly, it has been found that the combination of copper threads and UV-A radiation emitted by the optical fibers arranged near the copper threads makes it possible to significantly reduce or destroy the bacteria, in particular E. coli, contained in an aqueous medium. In addition, part of the copper ions released by the copper threads into the aqueous medium may be redeposited on the surface of the textile web, thus making it possible to maintain a copper stock for longer and therefore to ensure an antimicrobial effect over a longer period. duration. Thus, during the treatment process, the textile web may therefore have deposits of metal ions on the surface that were released by the metal threads during use.

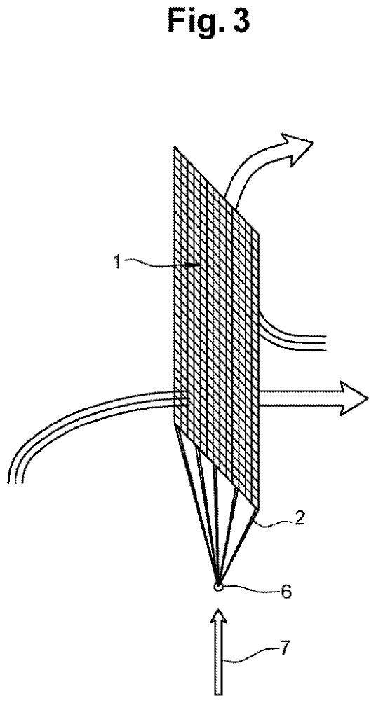

As may be seen from the curves in FIG. 4, the result of the invention is not the simple combination of the effects of the metal copper or silver and UV. There is a real increase and a synergy of the antibacterial effect of the textile web provided with copper and/or silver threads combined with UV radiation and in particular with UV-A radiation.

The protocol for the tests making it possible to obtain the curves C1-C7 is as follows: a standardized bacterial suspension of E. coli in an aqueous medium is produced. 180 mL of this solution are placed in a reactor and temporal measurements of the concentration of E. coli in the medium are carried out in the following cases:
curve C0: a textile web (dimensions 100*100 mm) made from fibers optics held by binding threads is immersed in the aqueous medium. The textile web is devoid of metallic threads and photocatalyst and is not connected to any light source. The aqueous medium is therefore not illuminated;
curve C1: the textile web used for the curve C0 is now connected to a light source generating UV-A radiation with a wavelength on the order of 365 nm. The aqueous medium is therefore illuminated with UV-A radiation;
curve C2: a textile web (dimensions 100*100 mm) according to any embodiment of the invention, incorporating metal threads but devoid of TiO₂photocatalyst, is immersed in the aqueous medium. The textile web is not connected to any light source, and the assembly is placed in the dark so as to avoid any light radiation. Each metal thread is specially formed of a thread consisting of copper and silver twisted with polyester textile thread;
curve C3: a textile web (dimensions 100*100 mm) according to any embodiment of the invention, incorporating metal threads also devoid of TiO₂ layer, is immersed in the aqueous medium. The assembly is also placed in the dark so as to avoid any light radiation. The textile web is not connected to any light source, and the assembly is also placed in the dark so as to avoid any light radiation. Each metal thread is a pure copper monofilament with a diameter of 0.1 mm;
curve C4: The textile web used for the curve C4 curve is similar to that used for the curve C2 except that each metal thread is obtained by assembling a polyamide thread impregnated with silver and a polyester. The textile web is immersed in the aqueous medium and the assembly is also placed in the dark so as to avoid any light radiation;
curve C5: the textile web used to obtain curve C2 is now connected to a light source, an LED, generating UV-A radiation with a wavelength of about 365 nm;
curve C6: the textile web used to obtain curve C3 is now connected to the light source generating UV-A radiation with a wavelength of about 365 nm;
curve C7: the textile web used to obtain curve C4 is now connected to the light source generating UV-A radiation with a wavelength on the order of 365 nm.

The medium is in recirculation. Measurements are taken every hour for 8 hours. In particular, the quantity of viable cultivable bacteria remaining in the medium is determined by counting the bacteria on a rich medium.

There is a real increase and a synergy of the antibacterial effect of the textile web provided with lateral emission optical fibers woven with silver and/or copper metallic threads (curves C5, C6 and C7) combined with UV radiation and in particular with UV-A radiation. The result of the invention is therefore not the simple combination of the effects of copper or silver metal and UV.

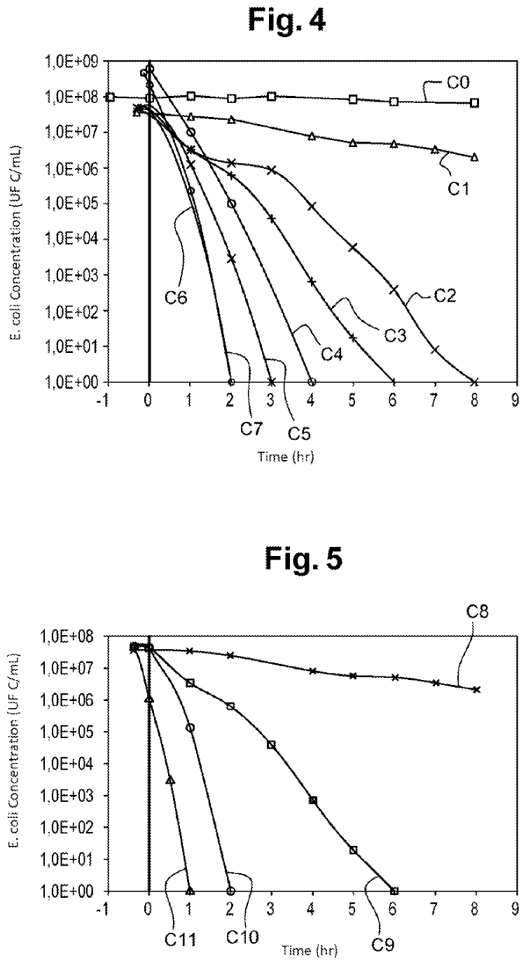

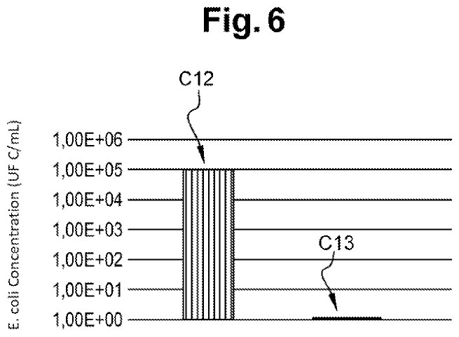

FIG. 5 also shows the notorious effect of the textile web of the invention based on copper threads and optical fibers diffusing UV-A radiation. The test protocol is identical to that described above. A standardized bacterial suspension of E. coli in an aqueous medium is made. 180 mL of this solution is placed in a reactor and time measurements of the concentration of E. coli in the medium are performed in the following cases:
curve C8: a textile web (dimensions 100*100 mm) based on optical fibers held by binding threads is immersed in the aqueous medium. The textile web is devoid of metal threads and of the TiO₂ particle and is connected to a light source generating UV-A radiation with a wavelength of about 365 nm;
curve C9: a textile web (dimensions 100*100 mm) according to any embodiment of the invention incorporating pure copper threads and devoid of a TiO₂layer, is immersed in the aqueous medium. The textile web is not connected to a light source and the assembly is placed in the dark so as to avoid any light radiation;
curve C10: the web used to obtain curve C9 is this time connected to a light source generating UV-A radiation with a wavelength of about 365 nm;
curve C11: a textile web (dimensions 100*100 mm) according to any embodiment of the invention integrating TiO₂ particles and pure copper threads, is immersed in the aqueous medium and is connected to the light source generating UV-A radiation with a wavelength of about 365 nm.

There is also an additional advantage of the antibacterial effect of the textile web coated with photocatalyst (TiO₂) according to the invention combined with UV-A radiation (curve C11) compared to a textile web of the invention without TiO₂ (Curve C10).

In a gaseous medium, for example in the surrounding air, the bacteria may be temporarily suspended in the air and the protocol used therefore aims to mimic this type of aerial bacterial contamination. An aerosol of a standardized E. coli bacterial solution is generated for 5 hours in continuous flow through a sealed device (chamber) containing the textile web of the invention integrating copper threads and a photocatalyst. At the outlet of the sealed device, the air flow containing the bacterial aerosol bubbles through a bottle containing an aqueous solution, making it possible to collect the bacteria still suspended in the air. Thus, curve C12 represents the quantity of viable cultivable bacteria present initially, determined by counting the bacteria on rich medium and curve C13 represents the quantity of bacteria counted at the end of the test after 5 hours under UV-A irradiation via the textile web of the invention. Under these experimental conditions, significant bacterial inactivation is observed when UV-A activates the TiO₂ compared to the results obtained with the control conditions.

This invention thus finds various applications such as the treatment of air in hospitals, of liquids, or of surfaces. The very structure of the textile web allows in particular easy installation in places where the supply of light radiation is not always easy, for example in a shoe for a disinfection phase via the connection of the textile web. to an LED generating UV radiation.

The treatment solution of the invention is essentially described in relation to the E. Coli bacterium, but it can also be implemented for the inactivation or elimination of other microorganisms such as those identified for copper and silver.

1. A system for the treatment of microorganisms comprising:
a textile web comprising optical fibers in warp and/or weft woven with binding threads in warp and/or weft, each of the optical fibers having invasive alterations along the fiber and allowing the emission of light propagating in the fiber at these alterations;
a light source arranged opposite one or both free ends of the optical fibers characterized in that the textile web further comprises metallic warp and/or weft threads woven with said binding threads, said metallic threads being based on a metal having a negative effect on the growth of microorganisms, the negative effect comprising the inactivation of the microorganisms or the reduction of their microbial activity;
and in that the light source generates a light beam comprising at least one wavelength in the visible or ultraviolet spectrum.

2. The treatment system according to claim 1, wherein the light source generates type A ultraviolet radiation or radiation with a wavelength of between 315 nm and 400 nm.

3. The treatment system according to claim 1, wherein the light source generates near UV visible radiation or with a wavelength of between 400 nm and 500 nm.

4. The treatment system according to claim 1, wherein the metal threads are made of material having antimicrobial properties.

5. The treatment system according to claim 1, wherein the metal threads are made of a material selected from the group comprising silver and copper.

6. The treatment system according to claim 1, wherein the textile web has two opposite visible surfaces, and in which optical fibers and metal threads held by binding threads are visible on the two opposing sides of the web.

7. The treatment system according to claim 1, wherein the textile web comprises a superposition of textile layers, each layer being formed of optical fibers and metal threads held by binding threads.

8. The treatment system according to claim 1, wherein the textile web has two opposite visible surfaces, and in which the optical fibers are visible on one of the surfaces, and the metal threads are visible on the other surfaces.

9. The treatment system according to claim 1, wherein the textile web has two opposite visible surfaces, and in which the optical fibers are visible on one of the surfaces, and the metal threads are visible on both surfaces.

10. The treatment system according to claim 1, wherein the textile web has two opposite visible surfaces, and in which the optical fibers are visible on the two surfaces, and the metal threads are visible on one of the two surfaces.

11. The treatment system according to claim 1, wherein the textile web further comprises a coating layer incorporating photocatalytic particles deposited on all or part of the optical fibers and/or on all or part of the binding threads, before weaving the optical fibers and the binding threads.

12. The treatment system according to claim 1, wherein the textile web further comprises a coating layer incorporating photocatalytic particles deposited on all or part of at least one of the surfaces of the fabric formed by the optical fibers and binding threads.

13. The treatment system according to claim 12, wherein the photocatalytic particles are formed from a material selected from the group consisting of titanium dioxide, zinc oxide, zirconium dioxide, and cadmium sulfide.

14. A method for treating microorganisms in a liquid or gaseous medium, comprising:
placing the textile web according to claim 1 in the said medium; and
illuminating the free ends of the optical fibers with said light source.
